(19) United States
(12) Patent Application Publication
(10) Pub. No.: US 2010/0185053 A1
(43) Pub. Date: Jul. 22, 2010

(54) Transverse surgical tunneling

(76) Inventor: Monika Elizabeth Hagen, Kreuzlingen (CH)

Correspondence Address: Monika Hagen;c/o Anne Zimmer, Steinbruchstrasse 9, Kreuzlingen, 8280, CH

(21) Appl. No.: 12/657,522

(22) Filed: Jan. 20, 2010

Related U.S. Application Data
(60) Provisional application No. 61/205,661, filed on Jan. 21, 2009.

(51) Int. Cl. A61B 1/04; A61M 25/00

(52) U.S. Cl. 600/114; 604/264

(57) ABSTRACT

The present invention is directed to a method for providing an access point for laparoscopic intra-abdominal surgery disconnected from the area of the ultimate intra-abdominal procedure. This method allows for the necessary skin incision(s) for insertion of the laparoscopic equipment to occur in a favorable area, transverse below one or more tissue layers to a secondary insertion point in a favorable area, and for the further insertion of the laparoscopic equipment. Favorable area selection affords the surgeon the ability, for example, to select a region of the patient that may result in a less obvious scar of the cutaneous layer of the abdominal wall for the first incision and optimal location in regards to mechanical stability of the muscular layer of the abdominal wall for the second incision. The invention further includes devices for performing such described method.

### PARENT CASE TEXT

This application claims the benefit under 35 U.S.C. 119(e) of U.S. provisional applications Ser. No. 61/205,661 filed Jan. 21, 2009, which is incorporated by reference herein in its entirety.

### BACKGROUND OF THE INVENTION

Transverse surgical tunneling for minimal invasive surgery relates to a new method for use in intra-abdominal surgery.

Intra-abdominal surgery has been traditionally performed by forming an incision in the abdominal wall in direct correlation to the region of surgery and operating upon internal body organs through such incision. This method of surgery invariably results in a certain trauma accompanied by a corresponding amount of postoperative recovery time and cosmetic impairment by scaring. Newer methods involving laparoscopy represent the access of a camera and multiple working instruments through cannulas placed in the abdominal wall in the most direct way (entering the abdominal cavity in 90 degree angle) to perform intra-abdominal surgery. For this method, the camera is traditionally placed in the region of the patient umbilicus with working channels placed in triangulation around the camera, targeting the region of procedure in the most direct way. Laparoscopy results in less abdominal wall trauma with less postoperative distress and better cosmetic outcomes compared to open surgery, but are more difficult and require special training for surgeons. Newer developments lead to single incision laparoscopic surgery during which multiple ports are inserted through a single skin incision or multiple instruments through a larger cannula at the umbilicus in order to improve the reduction of surgical trauma, recovery and cosmetics. Subsequent to laparoscopic surgery, there is a risk of the patient developing incision-based hernias and cosmetic impairments due to scaring. Incisions in the umbilical region might also include ischemic complications and deformation of the umbilicus.

Natural orifice surgery describes a novel technique during which the abdominal cavity is accessed through a natural orifice such as stomach, vagina, rectum, bladder, etc. This technique has been developed in animal and cadaveric models and has been conducted in selected human cases world-wide in early feasibility trials in a hybrid technique with laparoscopic support. This technique provides a preferable cosmetic outcome, with scaring in anatomical region not readily visible, as well as other potential advantages and reduced risk factors. However, execution of this technique is problematic as there is limited surgical instrumentation by which to most effectively conduct the necessary procedure.

The closest existing method to the present invention is ordinary laparoscopy and single incision laparoscopic surgery. During laparoscopy, trocars are placed directly through the abdominal wall in order to access the abdominal cavity. Normally, 3 to 7 trocars are used for procedure equipment including a camera—most often centrally located in order to work in triangulation—and laparoscopic instruments such as graspers, scissors, coagulating devices and many others. These trocars are usually placed in a triangle around the targeted area of surgery. Also, the trocars are placed in the most direct way through the entire abdominal wall. In other words, site of skin incision and entrance point into the abdominal cavity are at the same or almost the same level. No tunnelation is performed. During single incision surgery, a single skin incision—mainly around the belly-button—is performed and 2 to 4 trocars or special multichannel trocars are inserted through the abdominal wall.

During laparoscopy and single incision laparoscopy surgery, the point of skin incision and entrance to the abdominal cavity are in the same place without forming a subcutaneous tunnel resulting in a scar directly on the abdominal wall. The present invention represents a new method that allows the surgeon to specifically perform a first insertion point through a skin incision, for example in a cosmetically favorable place, and then to enter the abdominal cavity for inspection and surgery at a second and different place, preferably at a mechanically favorable location. Therefore the present is directed to transverse surgical tunneling for minimal invasive surgery, wherein a new method combines the advantages of traditional and laparoscopic surgery (feasibility, reproducibility and technical ease) with the advantages of a new technique such as natural orifice surgery with significantly improved cosmetic outcomes.

### SUMMARY OF THE INVENTION

The present invention is directed to a method for providing an access point for laparoscopic intra-abdominal surgery disconnected from the area of the ultimate intra-abdominal procedure. This method allows for the necessary skin incision(s) for insertion of the laparoscopic equipment to occur in first insertion point or site, transverse below one or more tissue layers to a secondary insertion point or site, and for the further insertion of the laparoscopic equipment. Favorable area selection affords the surgeon the ability, for example, to select a region of the patient that may result in a less obvious scar or already existing scars during the first incision.

A further embodiment of the present invention is to provide a method for forming an access point for laparoscopic intra-abdominal surgery disconnected from the area of the ultimate intra-abdominal procedure. This method allows for a surgeon to place the entrance incision through the abdominal wall in a location independent from the skin incision, wherein the abdominal wall incision is preferably placed in a mechanical favorable location.

A further embodiment of the present invention is to provide an access point for intra-abdominal surgery disconnected from the area of the intra-abdominal procedure in order to be able to place the skin incision in a favorable cosmetic area such as the pubic hair.

A further embodiment of the present invention is to provide access to intra-abdominal organs through a single skin incision placed within the pubic hair.

A further embodiment of the present invention includes devices for performing a method for forming an access point for laparoscopic intra-abdominal surgery disconnected from the area of the ultimate intra-abdominal procedure.

### DESCRIPTION OF THE SEVERAL VIEWS OF THE DRAWING(S)

The invention will be more easily understood by a detailed explanation of the invention including drawings. Accordingly, drawings, which are particularly suited for explaining the inventions, are attached herewith; however, it should be understood that such drawings are for descriptive purposes only and as thus are not necessarily to scale beyond the measurements provided. The drawings are briefly described as follows:

FIG. 1 is a side of the present invention wherein a steerable tunneling trocar is positioned through the cutaneous layer at a first insertion site of a patient undergoing laparoscopic surgery;

FIG. 2 is a side of the present invention wherein a steerable tunneling trocar is positioned through a first insertion site and a first tissue, wherein the leading edge of the trocar is then guided in a transverse tunneling heading;

FIG. 3 is a side of the present invention wherein a steerable tunneling trocar is positioned through a first insertion site and a first tissue, an intermediate transverse tunnel, and then the leading edge positioned for placement through a second, internal insertion site;

FIG. 4 is a side of the present invention wherein a steerable tunneling trocar is positioned through a first insertion site and a first tissue, an intermediate transverse tunnel, and through a second, internal insertion site;

FIG. 5 is a side of the present invention wherein a steerable tunneling trocar is positioned through a first insertion site and a first tissue, an intermediate transverse tunnel, and through a second, internal insertion site, wherein trocar used in placing the unit is removed leaving behind a positioned laparoscopic cannula;

FIG. 6 is a side of the present invention wherein a laparoscopic cannula is positioned through a first insertion site and a first tissue, an intermediate transverse tunnel, and through a second, internal insertion site, wherein an laparoscopic surgical device is inserted into the cannula;

FIG. 7 is a side of the present invention wherein a laparoscopic cannula is positioned through a first insertion site and a first tissue, an intermediate transverse tunnel, and through a second, internal insertion site, wherein an laparoscopic surgical device is inserted through the cannula and the distal end of which is now positioned to perform a surgical procedure;

FIG. 8 is magnified view of the leading edge of the trocar in accordance with the present invention wherein one or more optional blades are positioned on the leading edge of the trocar;

FIG. 9A is magnified view of the leading edge of the cannula in accordance with the present invention wherein mechanically responsive material is placed on the inside of the cannula such that upon activation, can be used to extend the cannula for perforation and tunneling;

FIG. 9B is magnified view of the leading edge of the cannula in accordance with the present invention as shown in FIG. 9A, wherein mechanically responsive material has been activated so as to extend the cannula for perforation and tunneling;

FIG. 10 is a cross sectional view along line X in FIG. 1 depicting tensioning lines spaced about the lumen of the cannula for purposed of steering the tunneling trocar during insertion;

FIG. 11 is a side of the present invention whereupon completion of the laparoscopic procedure through said second insertion site, the cannula is removed from the second insertion site and tissue of the second insertion site is durably connected thus closing the second insertion site; and,

FIG. 12 is a side of the present invention whereupon completion of the laparoscopic procedure through said first insertion site, the cannula is removed from the first insertion site and tissue of the first insertion site is durably connected thus closing the first insertion site.

### ASSIGNMENT OF COMPONENT NUMBERING

Cutaneous layer (Skin) 2, muscular tissue of abdominal wall 6, articulated tunneling trocar assembly 10, cannula 12, visualization means (i.e. camera) 13, trocar leading edge 14, visualization trocar shaft 15, trocar blade 16, cannula steering mechanism 17, cannula extension elements 18, tensioning lines 19, flexible shaft laparoscopic surgical device 20, tissue closure 22, laparoscopic tissue closure device 24, cannula valve 26.

### DETAILED DESCRIPTION OF THE INVENTION

While the present invention is susceptible of embodiment in various forms, there is shown in the drawings and will hereinafter be described a presently preferred embodiment of the invention, with the understanding that the present disclosure is to be considered as an exemplification of the invention, and is not intended to limit the invention to the specific embodiment illustrated.

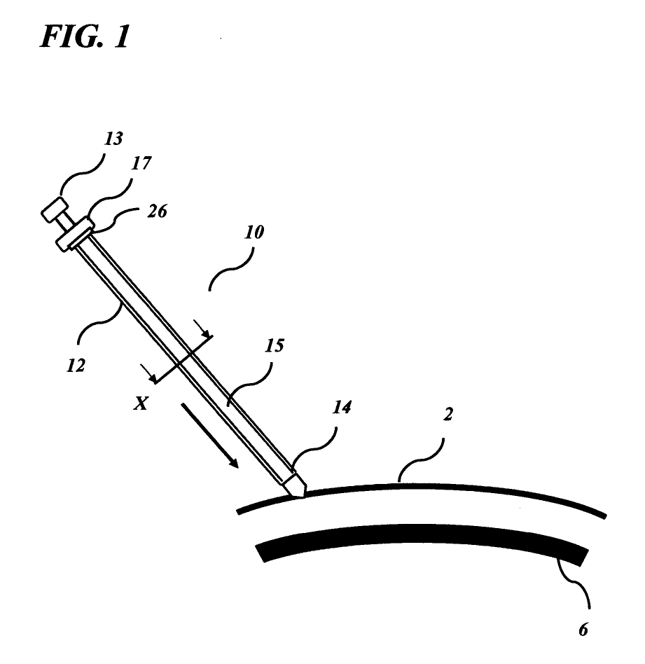

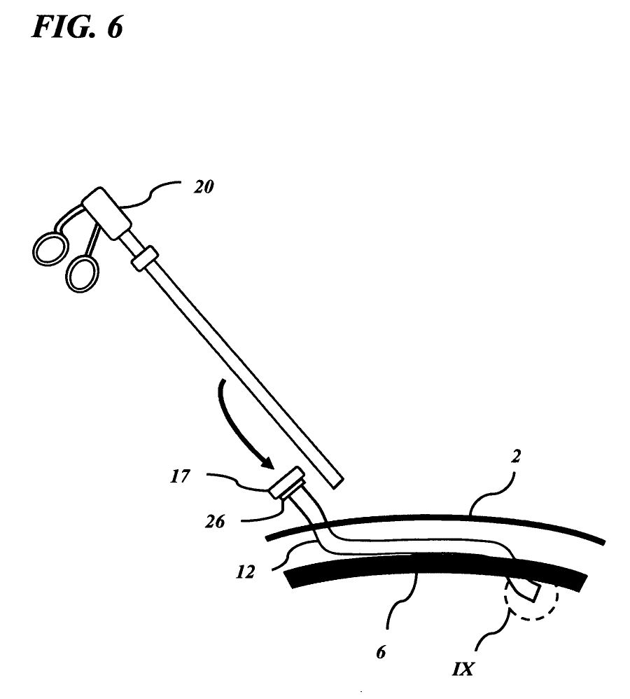

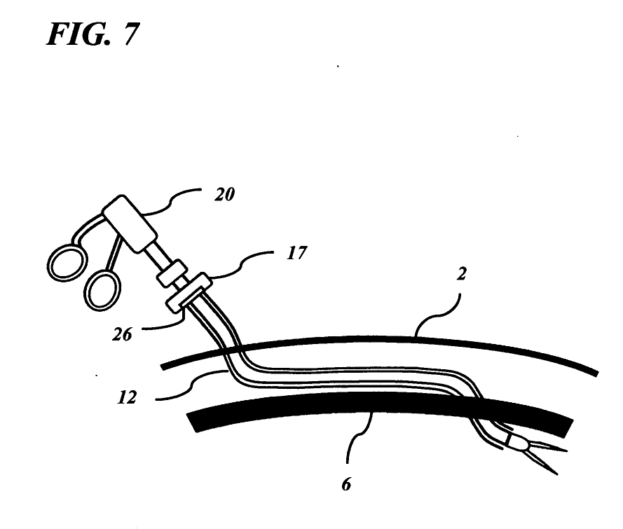

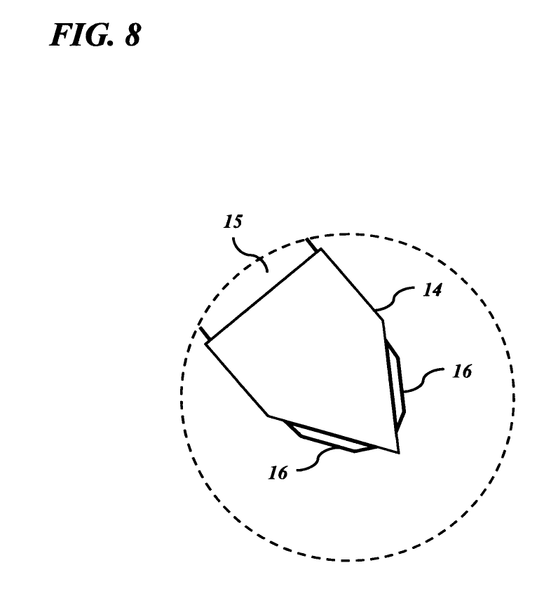

Referring more specifically to the figures, for illustrative purposes the present invention is embodied in the method and apparatus generally shown in FIG. 1 through FIG. 12.

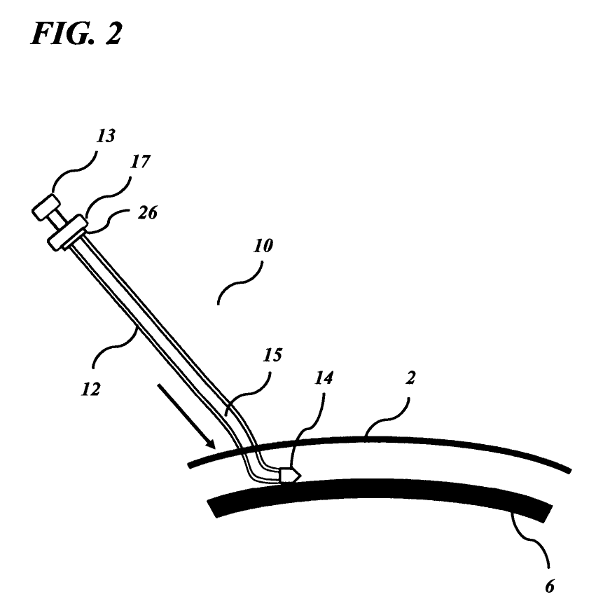

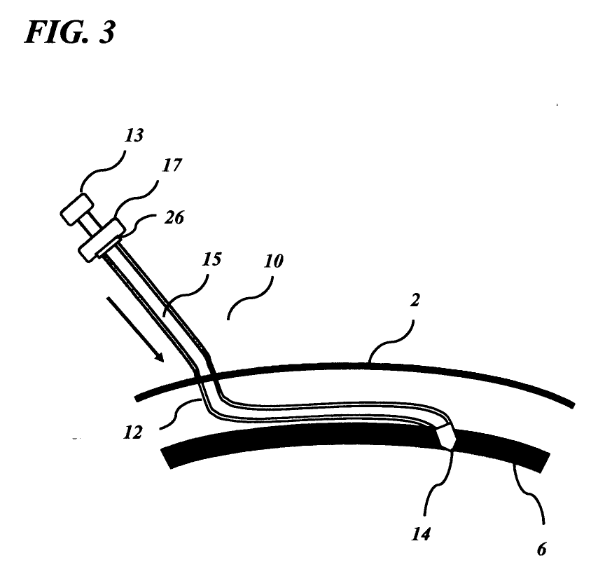

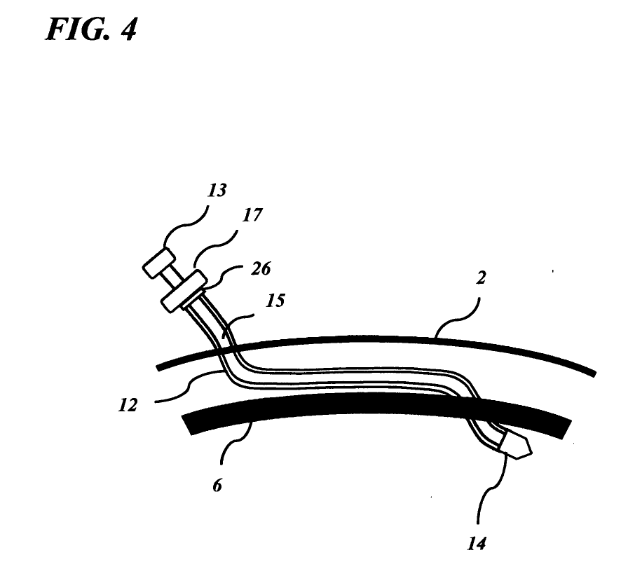

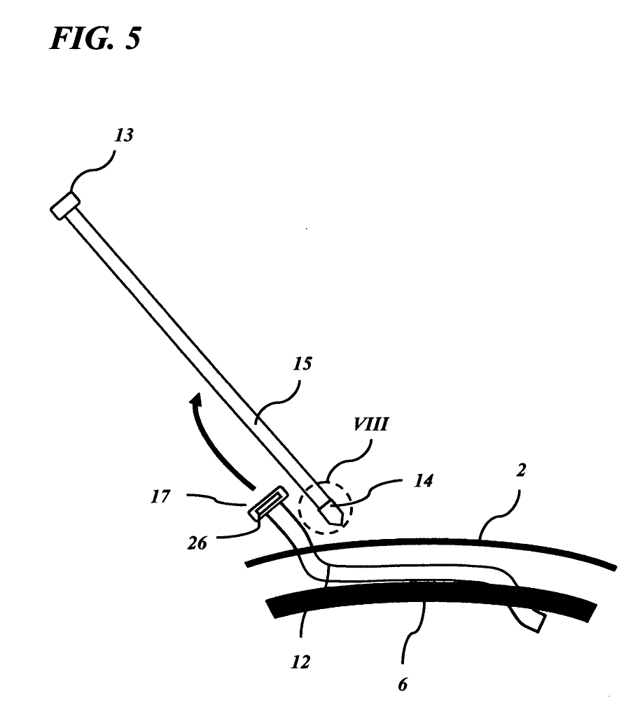

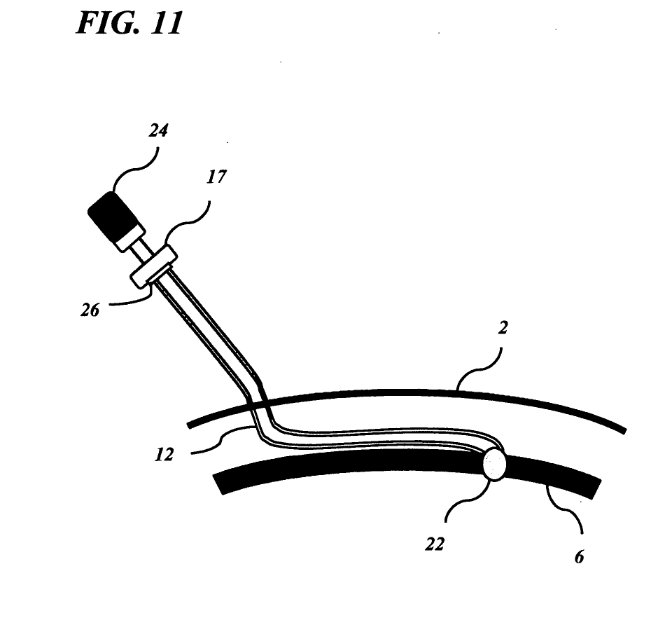

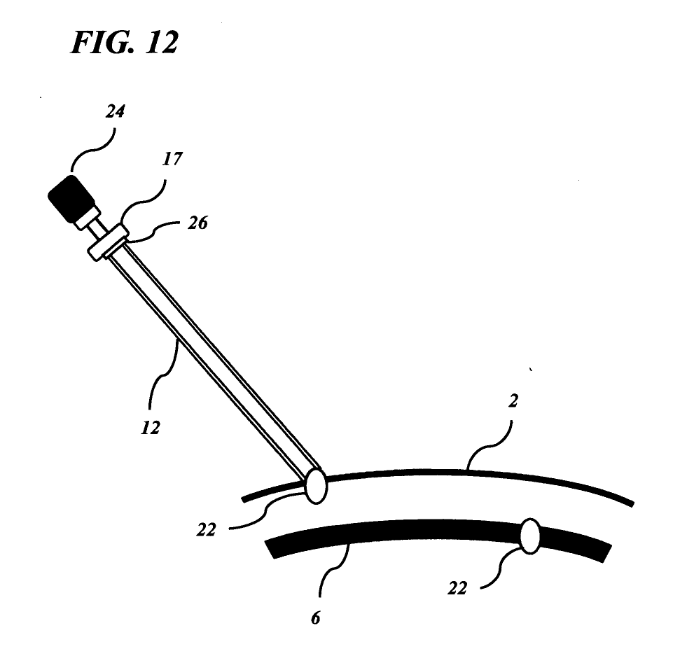

Turning to FIG. 1 through 7, a method for use in intra-abdominal surgery comprises, in accordance with the present invention the steps of (a) placing a skin incision 2 in a cosmetically favorable location such as the pubic hair (FIG. 1), (b) creating a tunnel in the subcutaneous layer with a cannula 12 under the vision imparted by visualization trocar shaft 14 of an endoscope inserted into that cannula 12 (FIGS. 1, 2), (c) introducing the cannula 12 with an inserted endoscope into the subcutaneous tunnel (FIGS. 1, 2), (d) manipulation of the cannula 12 from outside the patient to incise the abdominal wall 6 through the muscle, linea alba or any other desired location (FIGS. 3, 4), (e) using the endoscope to visually inspect internal body tissues in an abdominal cavity of the patient (FIG. 4), (f) executing a surgical operation on natural body tissues by surgical instruments 20 passed through the cannula 12 (FIG. 5-7) (g) withdrawing the laparoscopic instruments 20 and the cannula 12 through the incisions, and (h) sequentially closing the insertion sites (FIGS. 11, 12).

Due to the compound contour path by which the first and second insertion sites may be placed, preformed cannulas 12 such as ordinary trocars are contraindicated for this new method. Preformed cannulas 12 are not able to perform a smooth s-shaped movement needed to form a subcutaneous tunnel connecting two separate insertion points or sites. This tunnel is an essential part of this method since point of entrance through the skin 2 and entrance through the abdominal wall 6 to the abdominal cavity are not at the same location. The longer the subcutaneous is tunnel is, the more important is the aspect of having a cannula with a flexible proximal and distal end with a semi-flexible shaft. Application of ordinary rigid laparoscopic trocars to perform subcutaneous tunnel is limited by an extremely short traverse distance and trauma to the skin 2 above the tunnel being stretched. Flexible catheters or cannulas 12 in the other hand do not deliver the stiffness that is needed to form a stable subcutaneous tunnel safely and controlled in dimension and direction. Also, entrance to the abdominal cavity requires a certain amount of force that will not be deliverable through a flexible device.

The present new method is performed by sequential progression through the following steps:

A skin incision 2 is placed at a point of first insertion in any cosmetically or medically favorable place of the patient's body. Cosmetically favorable points or sites of first insertion include those that are not aesthetically or obviously visible (i.e. the pubic hair, the arm pit, inguinal area). Medically favorable points or sites of first insertion include those that may set apart from of a region of previous trauma, such as previous scarring from prior surgical procedures or as a result of burns.

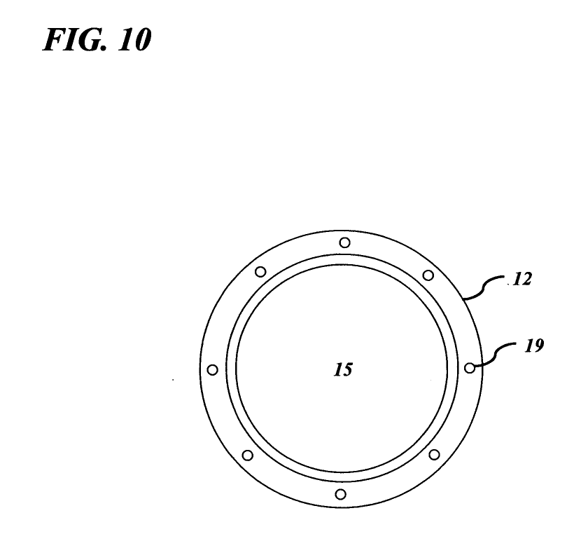

An articulated tunneling trocar assembly 10 is introduced through the cutaneous layer 2 into the subcutaneous layer and is advanced transversely in the subcutaneous layer until the preferred point of entrance into the abdominal cavity is reached. This point can differ dependent of area of surgery, scars from previous surgery and other parameters. More than one articulated tunneling trocar assembly 10 might be used at the same time in order to perform laparoscopic surgery. Point of skin 2 incision for first insertion site and entrance of the inner layer of the abdominal wall 6 can differ for each cannula. Generally, entrance through the inner layer of the abdominal wall 6 can be placed in different locations and the mechanically most favorable can be chosen. Access through the midline (Linea alba) or muscles are possible. The tunnelation of the articulated tunneling trocar assembly 10 through the subcutaneous layer will be performed under vision of a camera 13 through trocar shaft 15 in cannula 12 looking out through the tip of the trocar leading edge 14. As the tunneling trocar assembly 10 is advanced, control means such as exemplified by tensioning lines 19 in cannula 12 lumen (FIG. 10) are tightened or loosed so as to induce deviation of the cannula 12, and the overall articulated tunneling trocar assembly 10 in the direction of increased tension. Therefore, by alternating tension of the tension lines 19 in cannula 12, the articulated trocar assembly 10 is able to follow to a surgeon defined path from the first insertion point, transversely through the subcutaneous or other tissue layer, and to then achieve the second insertion point.

Once the point of desired perforation of the inner layer of the abdominal is reached, the tip of the cannula 12 will be bent towards the inner layer of the abdominal wall 6.

The inner layer of the abdominal wall is then perforated under vision from a camera 13 inside the cannula 12.

A blunt or sharp dissector blade 16 on the trocar leading edge 14 within cannula 12 will be used for the formation of the incision in the inner layer of the abdominal wall 6. The trocar leading edge 14 will be forwarded together with the tip of the cannula in order to perforate the inner layer of the abdominal wall 6. The trocar leading edge 14 maybe of pyramidal or conical penetration form with a symmetrical or asymmetrical cross section.

After positioning of one or more cannulas, laparoscopic instruments including camera and working instruments or a flexible endoscope with instruments will be inserted in order to perform surgery

Proximal end of cannula: The proximal end of the cannula 12 will be flexible with a steering mechanism 17. In that sense, the cannula 12 can smoothly perform a S-shape entrance through the skin, subcutaneous tissue and the inner layer of the abdominal wall (Muscle, midline, other fascia). The material might be rubber or any other flexible material with metal wires (or any other strong material) inserted as tension lines 19 for steering the cannula 12 from outside the patient. This part of the cannula 12 will also have the ability to straighten after successful insertion in order to be able to introduce rigid instruments for surgery through the cannula. Furthermore, it is advantageous that the cannula 12 has the smallest possible working outside diameter in order to avoid collisions of plural cannulas outside the patient if more than one cannula is placed close to each other.

The cannula 12 will have a valve 26 inside the proximal end. The valve 26 will prevent air leaks when camera and/or instruments are inserted. This valve 26 needs to be elastic and will be made out of rubber or other elastic material. The valve 26 will automatically adjust to close around instruments smaller than the inner diameter of the cannula 12. This mechanism might be achieved by different layers of elastic material

The shaft 15 of the trocar will be made out of flexible, semi-rigid or rigid material. The length of the shaft will mainly determine the length of the entire cannula 12. Different lengths of cannula 12 will be necessary to work with different locations of skin 2 incision and entry through the abdominal wall 6 into the abdominal cavity.

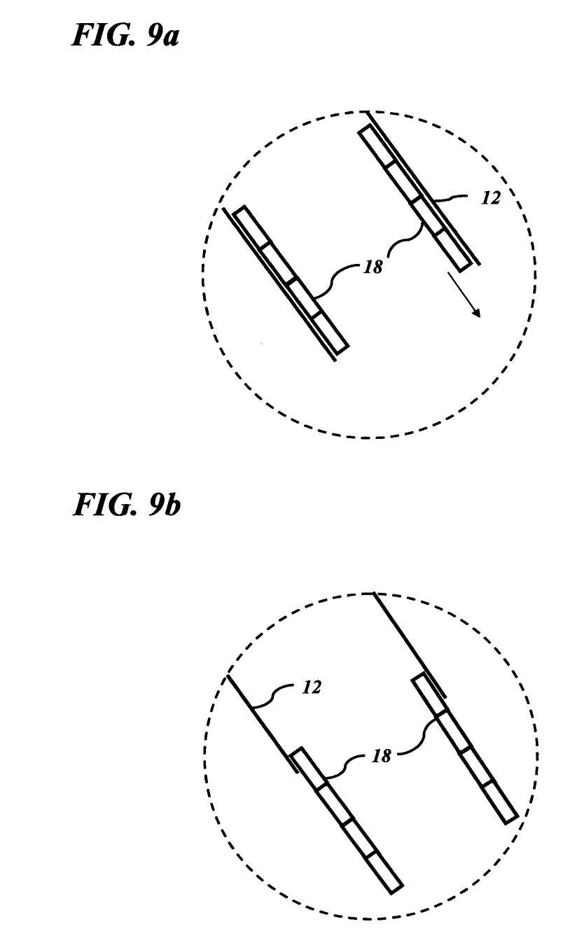

The distal end of the cannula 12 will be similar to the proximal end: This part of the cannula 12 again plays an important role in performing the s-shaped entrance to the abdominal cavity. Therefore, the distal end of the cannula 12 will be made of rubber or any other flexible material with metal wires (or any other strong material) inserted as tensioning line 19 for steering this part of the cannula from the outside. This part of the cannula 12 will also have the ability to straighten after successful insertion in order to be able to introduce rigid instruments for surgery through cannula 12. As per FIGS. 9A and B, an interactive tissue/cannula extension element 18 made of a rigid material (i.e. polymeric plastic) is positioned inside the cannula 12 tip with the ability to be brought outside to lengthen the cannula 12 during insertion. The cannula extention element 18 mechanism will be controlled from external to the patient. Furthermore, the distal end of the cannula 12 will have the ability to expand in order to enter the abdominal cavity from the subcutaneous layer. This mechanism will be followed by different layers of the outside trocar material that will be pushed forward in order to advance the distal tip of the cannula.

A dilator in the form of a trocar leading edge 14 will be introduced into the cannula 12 to create access through the inner layer of the abdominal wall. The trocar leading edge 14 will be hollow for insertion of a camera 17. The tip of the trocar leading edge 14 will be made out of transparent material so that a camera 17 inserted into the trocar leading edge 14 will be able to provide visual control of the insertion procedures. The trocar leading edge 14 will have a coned tip with blunt or sharp blades 16 for dissection of the inner layer of the abdominal wall for the second insertion point or site. The blades 16 will dissect the inner layer of the abdominal wall 6 by means including mechanical action by an external manipulation of the trocar shaft 15 inside cannula 12 and pressure might be applied mechanically from external of the patient's abdominal wall 6. The material of the trocar leading edge 14 has to be durable at the tip so as to allow dissection of an entry point at the inner layer of the abdominal wall and has to be transparent such as plastic for visualization by camera 17 there through. The trocar shaft 15 affixed to the trocar leading edge 14 has to be made of flexible material in order to be able to perform cannula 12 s-shape. The shaft 15 material should have the ability to transmit external application of force to the trocar leading edge 14 in order to allow successful dissection.

The method of transverse tunnel may be reversed at such time the laparoscopic surgical procedure is complete (FIGS. 11, 12). A tissue closure device 24 may be inserted through cannula 12 as cannula retention elements 18 are released so that tissue closures 22 can be first made in the second insertion point through the abdominal wall 6. Cannula 12 may then be reversed further using the cannula steering mechanism 17 so as to move cannula 12 back through the transverse tunnel which was first formed. The first insertion point or site may then have tissue closures 22 applied so as to close the first insertion point in skin 2.

The cannula will have a small channel for gas (not shown). A small connector at the proximal end of the cannula will be provided for connection to a gas pump. The gas channel will lead gas from the proximal end of the cannula through the wall of the cannula's shaft to the distal end and inside the abdominal cavity. By having this connection and the gas channel, pneumoperitoneum can be installed, maintained and controlled.

The following patents are incorporated by reference in their entireties:

| | |
|---|---|
| U.S. Pat. No. 3,613,684 | Trocar catheters |
| U.S. Pat. No. 4,863,430 | Introduction set with flexible trocar with |
| | curved cannula |
| U.S. Pat. No. 5,041,085 | Percutaneous lockable sleeve catheter |
| U.S. Pat. No. 5,205,830 | Catheter assembly |
| U.S. Pat. No. 5,263,937 | Trocar with profile to reduce insertion force |
| U.S. Pat. No. 5,279,551 | Trocar catheter |
| U.S. Pat. No. 5,468,248 | Endoscopic inflatable retraction devices for |
| | separating layers of tissue |
| U.S. Pat. No. 5,509,909 | Bent chest tube assembly |
| U.S. Pat. No. 5,609,562 | Visually directed trocar and method |
| U.S. Pat. No. 5,800,409 | Flexible inflow/outflow cannula |
| U.S. Pat. No. 6,007,483 | Surgical method for developing an anatomic |
| | structure |
| U.S. Pat. No. 6,352,530 | Slideable cannula and method of use |
| U.S. Pat. No. 6,554,793 | Flexible trocar with an upturning tube system |
| U.S. Pat. No. 6,669,708 | Devices, systems and methods for creating |
| | sutureless on-demand vascular anastomoses |
| | and hollow organ communication channels |
| U.S. Pat. No. 7,326,197 | Method and apparatus for chest drainage |
| U.S. Pat. No. 7,344,547 | Laparoscopic instruments and trocar systems |
| | and related surgical method |
| U.S. Pat. No. 7,398,781 | Method for subxiphoid endoscopic access |
| US 20020161353 | Steerable catheter with reinforced tip |

Beneficially, cannulas 12 should have the ability to be straightened in order to have the option to use rigid laparoscopic instruments.

The inner layer of abdominal wall 6, as used herein, shall include either the muscles or the midline (Linea alba)

Although the description above contains many details, these should not be construed as limiting the scope of the invention but as merely providing illustrations of some of the presently preferred embodiments of this invention. Therefore, it will be appreciated that the scope of the present invention fully encompasses other embodiments, which may become obvious to those skilled in the art. In the appended claims, reference to an element in the singular is not intended to mean “one and only one” unless explicitly so stated, but rather “one or more.” All structural, chemical, and functional equivalents to the elements of the above-described preferred embodiment that are known to those of ordinary skill in the art are expressly incorporated herein by reference and are intended to be encompassed by the disclosure and present claims. Moreover, it is not necessary for a device or method to address every problem sought to be solved by the present invention, for it to be encompassed by the disclosure and present claims. Furthermore, no element, component, or method step in the present disclosure is intended to be dedicated to the public regardless of whether the element, component, or method step is explicitly recited in the claims. No claim element herein is to be construed under the provisions of 35 U.S.C. 112, sixth paragraph, unless the element is expressly recited using the phrase “means for.”

What is claimed is:

1) A method for performing endoscopic surgery comprising:
a. forming a first insertion site through a first tissue layer;
b. inserting a trocar/cannula assembly into said first insertion site;
c. traversing said trocar/cannula assembly beneath said first tissue layer;
d. displacing tissue of a second tissue layer using said trocar/cannula assembly at a second insertion site;
e. continuing to insert said trocar/cannula assembly through said second tissue layer;
f. releasing said cannula from said trocar assembly;
g. removing said trocar assembly allowing said cannula to remain in place;
wherein said cannula simultaneously traverses said first and second tissue layers; and
wherein said first insertion site and said second insertion site are displaced laterally by a distance of at least 1.0 cm.

2) A method for performing endoscopic surgery as in claim 1, wherein said first insertion site is located in an anatomical region wherein scarring is not readily observable.

3) A method for performing endoscopic surgery as in claim 2, wherein said anatomical region is the pubic hair.

4) A method for performing endoscopic surgery as in claim 1, wherein said second insertion site is the muscular layer of the abdominal wall.

5) A method for performing endoscopic surgery as in claim 1, wherein said primary incision site and said secondary site are displaced laterally within the range of 2.0 cm and 10 cm.

6) An articulated trocar assembly, said trocar assembly comprising:
a) a trocar leading edge;
b) a trocar shaft affixed to said trocar leading edge;
c) a cannula through which said trocar shaft is contained;
d) a cannula steering mechanism;
wherein said cannula steering mechanism is operatively attached to said cannula such that
application of force to the articulated trocar assembly combined with input from an operator induces the articular trocar assembly to follow a desired path,
whereupon placement of the articulated trocar assembly, said cannula is released to retain said operator selected path and said trocar shaft ant leading edge are removed.

7) An articulated trocar assembly as in claim 6, wherein said trocar leading edge further comprises one or more blades.

8) An articulated trocar assembly as in claim 7, wherein said blades are capable of cutting tissue.
